(19) United States
(12) Patent Application Publication
(10) Pub. No.: US 2011/0257681 A1
(43) Pub. Date: Oct. 20, 2011

(54) Surgical Forceps Including Geared Blade Reverser Mechanism

(75) Inventors: Arlen J. Reschke, Longmont, CO (US); Jeffrey M. Roy, Boulder, CO (US)

(73) Assignee: TYCO Healthcare Group LP

(21) Appl. No.: 12/763,909

(22) Filed: Apr. 20, 2010

(51) Int. Cl. A61B 17/28

(52) U.S. Cl. 606/206

(57) ABSTRACT

A forceps includes first and second shafts each having a jaw. At least one jaw is moveable from an open to a closed position. At least one jaw includes a blade slot for reciprocation of a blade therethrough. An actuation assembly disposed within one shaft is configured for translating the blade between a retracted and at least one extended position. The blade extends into the blade slot in the extended position(s). The actuation assembly includes an actuator extending from the shaft and engaging an actuator rack. A gear is coupled to the actuator rack and is rotatably disposed on a pin. A blade rack is coupled to the gear and has the blade disposed at an end thereof. A gear box is configured to house the gear and at least a portion of the actuator and blade racks. The actuator and blade racks are translatable through the gear box.

### BACKGROUND

The present disclosure relates to a surgical forceps and, more particularly, to a surgical forceps including a geared blade reverser mechanism.

### TECHNICAL FIELD

A forceps is a plier-like instrument which relies on mechanical action between its jaws to grasp, clamp and constrict vessels or tissue. Electrosurgical forceps utilize both mechanical clamping action and electrical energy to affect hemostasis by heating tissue and blood vessels to coagulate and/or cauterize tissue. Certain surgical procedures require more than simply cauterizing tissue and rely on the unique combination of clamping pressure, precise electrosurgical energy control and gap distance (i.e., distance between opposing jaw members when closed about tissue) to “seal” tissue, vessels and certain vascular bundles.

Typically, once a vessel is sealed, the surgeon has to accurately sever the vessel along the newly formed tissue seal. Accordingly, many vessel sealing instruments have been designed which incorporate a knife or blade member which effectively severs the tissue after forming a tissue seal. However, imprecise separation of tissue may result from, for example, misalignment of the blade member with respect to the sealing line. Blade misalignment may also result in blade overload and/or blade fracture, which may pose problems to the user.

### SUMMARY

In accordance with the present disclosure, a forceps is provided. The forceps includes first and second shaft members each having a jaw member disposed at a distal end thereof. One or both of the jaw members is moveable from an open position to a closed position for grasping tissue therebetween. One or both jaw members includes a blade slot extending longitudinally therethrough. The blade slot is configured for reciprocation of a blade therethrough. An actuation assembly is disposed within one of the shaft members and is configured for selectively translating the blade between a retracted position and an extended position. The blade extends partially, or entirely, through the blade slot in the extended position. The actuation assembly includes an actuator extending from the shaft member and mechanically engaging an actuator rack. A gear member is mechanically coupled to the actuator rack and has a pin disposed therethrough. The gear member is rotatable with respect to the pin. A blade rack is mechanically coupled to the gear member and has a blade disposed at a distal end thereof. A gear box is also included. The gear box is configured to house the gear member and a portion of the actuator rack and the blade rack. The actuator rack and the blade rack are longitudinally translatable through the gear box. The gear box maintains the gear member, the actuator rack, and the blade rack in a fixed spatial relation relative to one another.

In one embodiment, translating the actuator proximally translates the blade distally to the extended position.

In another embodiment, the gear box is made from a metal.

In another embodiment, the pin is fixedly engaged within the gear box such that the gear member is rotatably secured therein.

In yet another embodiment, the gear member includes two or more teeth disposed along an outer surface thereof. The gear teeth are configured for engagement with teeth disposed along both the actuator rack and the blade rack.

In still another embodiment, the gear box includes one or more guide tracks defined therein. The guide track(s) is configured to guide longitudinal translation of the actuator rack and/or the blade rack through the gear box. Each guide track may include a pair of recesses defined within opposing sides of the gear box and the actuator rack and/or the blade rack may include a pair of protrusions extending from opposite longitudinal sides thereof such that the protrusions engage the recesses to thereby guide the translation of the actuator rack and/or the blade rack through the gear box. Alternatively, each guide track may include a pair of protrusions extending inwardly from opposing sides of the gear box and the actuator rack and/or the blade rack may include a pair of recesses defined therein along opposite longitudinal sides thereof such that the protrusions engage the recesses to thereby guide the translation of the actuator rack and/or the blade rack through the gear box.

In yet another embodiment, the actuation assembly includes one or more biasing members for biasing the blade in the retracted position.

### BRIEF DESCRIPTION OF THE DRAWINGS

Various embodiments of the subject instrument are described herein with reference to the drawings wherein:

FIG. 1 is a side, perspective view of a forceps according to an embodiment of the present disclosure;

FIG. 2 is a side, perspective view of the forceps of FIG. 1 with a portion of a handle removed to show the internal components therein;

FIG. 3 is a top view of a jaw member of the forceps of FIG. 1;

FIG. 4 is a schematic illustration of an actuation assembly of the forceps of FIG. 1, with parts separated;

FIG. 5 is a schematic illustration of another embodiment of an actuation assembly of the forceps of FIG. 1; and

FIG. 6 is a rear, cross-sectional view of the actuation assembly of FIG. 5.

### DETAILED DESCRIPTION

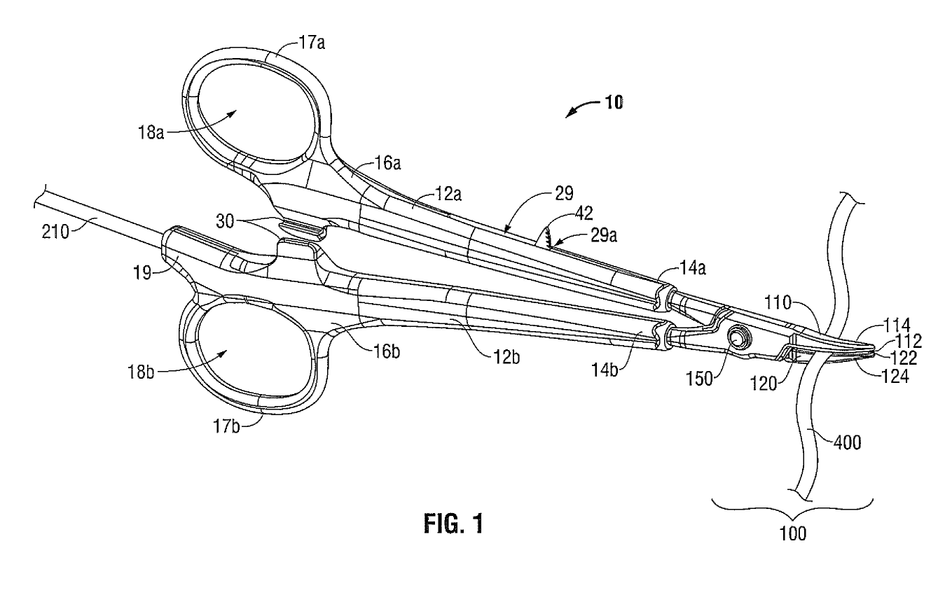

Referring initially to FIG. 1, a forceps 10 includes two elongated shafts 12a and 12b each having a proximal end 16a and 16b and a distal end 14a and 14b, respectively. In the drawings and in the descriptions which follow, the term “proximal,” as is traditional, will refer to the end of the forceps 10 that is closer to the user, while the term “distal” will refer to the end that is further from the user.

The forceps 10 includes an end effector assembly 100 attached to distal ends 14a and 14b of shafts 12a and 12b, respectively. As explained in more detail below, the end effector assembly 100 includes a pair of opposing jaw members 110 and 120 that are pivotably connected about a pivot pin 150.

Each shaft 12a and 12b includes a handle 17a and 17b disposed at the proximal end 16a and 16b thereof. Each handle 17a and 17b defines a finger hole 18a and 18b therethrough for receiving a finger of the user. As can be appreciated, finger holes 18a and 18b facilitate movement of the shafts 12a and 12b relative to one another which, in turn, pivots the jaw members 110 and 120 from an open position wherein the jaw members 110 and 120 are disposed in spaced-apart relation relative to one another to a closed position (FIG. 1) wherein the jaw member 110 and 120 cooperate to grasp tissue 400 therebetween.

A ratchet 30 may be included for selectively locking the jaw members 110 and 120 relative to one another at various positions during pivoting. Ratchet 30 may include graduations or other visual markings that enable the user to easily and quickly ascertain and control the amount of closure force desired between the jaw members 110 and 120.

With continued reference to FIG. 1, one of the shafts, e.g., shaft 12b, includes a proximal shaft connector 19 that is designed to connect the forceps 10 to a source of electrosurgical energy such as an electrosurgical generator (not shown). Proximal shaft connector 19 secures an electrosurgical cable 210 to the forceps 10 such that the user may selectively apply electrosurgical energy as needed.

As mentioned above, the two opposing jaw members 110 and 120 of the end effector assembly 100 are pivotable about pivot pin 150 from the open position to the closed positions for grasping tissue 400 therebetween. Jaw member 110 includes an insulated outer housing 114 that is dimensioned to mechanically engage an electrically conductive sealing surface 112 of jaw member 110. Similarly, jaw member 120 includes an insulated outer housing 124 that is dimensioned to mechanically engage an electrically conductive sealing surface 122 of jaw member 120. Electrically conductive sealing surfaces 112 and 122 are opposed to one another, such that, upon activation, electrosurgical energy may be supplied to the electrically conductive sealing surfaces 112 and 122 for sealing tissue disposed between the jaw members 110 and 120.

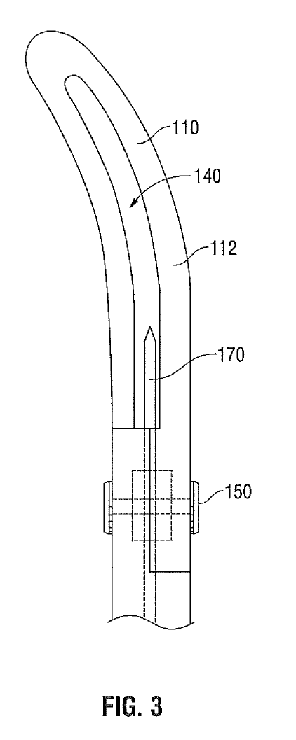

As best seen in FIG. 3, jaw member 110 includes a blade slot, or blade channel 140 extending therethrough. The blade channel 140 is configured for reciprocation of a cutting mechanism, e.g., a blade 170, therethrough. As shown, blade channel 140 is defined completely within jaw member 110. However, blade channel 140 may be formed when two opposing blade channels defined within jaw members 110 and 120 come together upon pivoting of the jaw members 110 and 120 to the closed position. Further, the blade channel 140 may be configured to facilitate and/or enhance cutting of tissue during reciprocation of the cutting blade 170 in the distal direction.

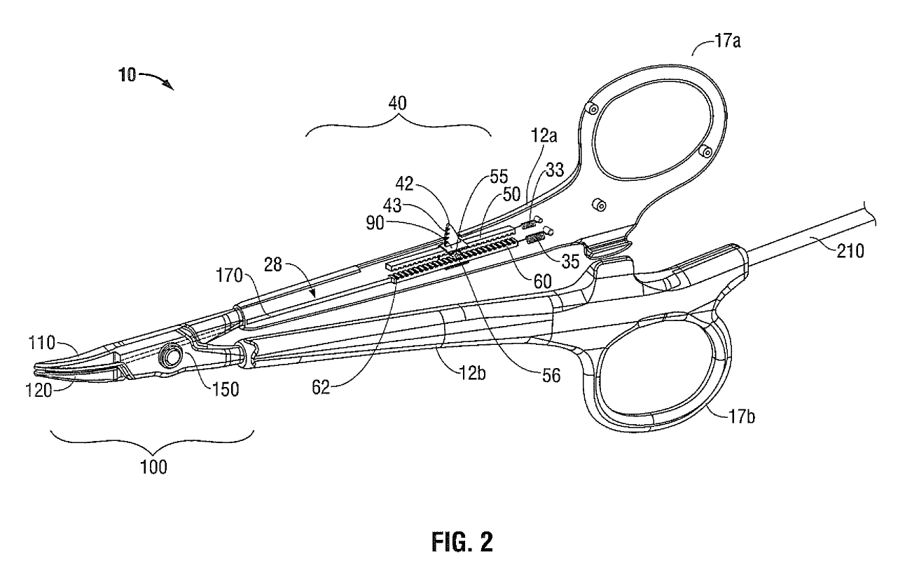

Referring now to FIG. 2, the arrangement of shaft 12a is slightly different from shaft 12b. As shown in FIG. 2, shaft 12a is hollow to define a chamber 28 therethrough that is configured to house an actuation assembly 40 therein. Actuation assembly 40 includes an actuator 42, an actuator gear rack 50, a gear member 55, and a blade gear rack 60. The actuator 42 includes a finger tab 43 that extends through a slot 29 defined within shaft 12a such that a user may engage the finger tab 43 to translate the actuator 42 longitudinally. Actuator 42 is attached to actuator rack 50. More specifically, actuator 42 may be integral with, or mechanically engaged to actuator rack 50 via any suitable mechanism. Blade rack 60 includes blade 170 disposed at a distal end 62 thereof. Blade 170 may be integral with blade rack 60, or may be attached thereto by other suitable mechanisms, e.g., by a plurality of pins (not shown) disposed through both blade 170 and blade rack 60.

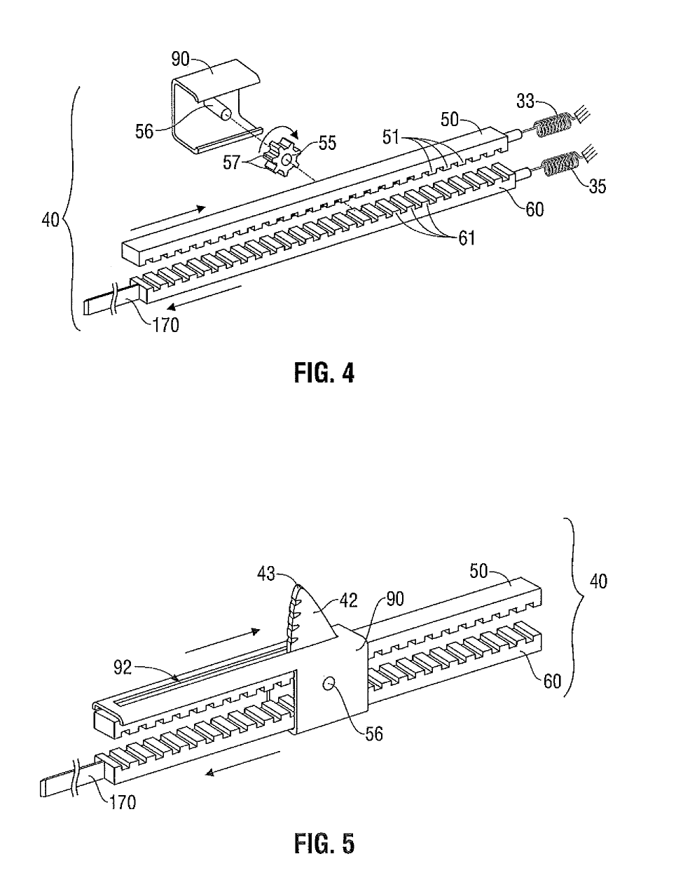

A gear member 55 is interdisposed between actuator rack 50 and blade rack 60, as best seen in FIGS. 2 and 4. Gear member, or pinion gear 55, is mounted on a pin 56, such that pinion gear 55 is rotatable with respect to pin 56. Teeth 57 disposed radially about pinion gear 55 mechanically mesh with teeth 51 and 61 of racks 50 and 60, respectively, such that pinion gear 55 converts proximal motion of actuator rack 50 into distal translation of blade rack 60 and vice versa. More particularly, when a user pulls finger tab 43 in a proximal direction through slot 29 of shaft 12a, actuator rack 50 is translated proximally which, in turn, rotates pinion gear 55 in a clockwise direction. Rotation of pinion gear 55 in a clockwise direction urges blade rack 60 to translate distally, thereby translating blade 170 distally through blade channel 140 of jaw members 110 and 120 to cut tissue 400 grasped therebetween. Put more generally, finger tab 43 is moveable between a distal position, whereby blade 170 is moved to a retracted position such that blade 170 is disposed completely within shaft 12a, and a proximal position, whereby blade 170 is moved to an extended position such that blade 170 extends into blade channel 140 of jaw members 110 and 120.

It is envisioned that multiple gears or gears with different gear ratios may be employed to reduce surgical fatigue which may be associated with advancing the blade 170. In addition, it is contemplated that racks 50 and 60 may be of different lengths to provide additional mechanical advantage for advancing the blade 170 through tissue.

Referring now to FIGS. 2, 4 and 5, gear box 90 is disposed within chamber 28 of shaft 12a and houses pinion gear 55 therein. More specifically, pin 56 is fixedly disposed within gear box 90, with pinion gear 55 rotatably mounted thereon. Actuator rack 50 and blade rack 60 are partially disposed through gear box 90. Gear box 90 is configured to maintain the fixed spatial relationship between pinion gear 55, actuator rack 50, and blade rack 60, while allowing pinion gear 55 to rotate with respect to gear box 90 and allowing actuator rack 50 and blade rack 60 to translate longitudinally through gear box 90. Further, gear box 90 guides and supports actuator rack 50 and blade rack 60 during translation through gear box 90. This configuration, wherein gear box 90 houses pinion gear 55, and at least a portion of the actuator and blade racks 50 and 60, respectively, helps prevent binding of gear 55 to actuator rack 50 and/or blade rack 60, which may occur where the racks 50, 60 are in too close a proximity to the gear member 55. This configuration also helps prevent disengagement and/or misalignment of the racks 50, 60, which may occur if the racks 50, 60 become spaced apart from the gear member 55.

Gear box 90 may be made of metal, or any other adequately rigid material. While the remainder of the actuator assembly 40 may be made from a plastic, or less rigid material, gear box 90 is made of metal or like material to prevent flexing when under a load such that the vertical and lateral spaced relationship between the racks 50, 60 and the pinion gear 55 is maintained throughout translation of the blade 170 between the retracted position and the extended position. As can be appreciated, maintaining the spatial relation between the racks 50, 60 and pinion gear 55 allows for smooth, accurate translation of blade 170 through blade channel 140 to cut tissue 400 disposed between jaw members 110 and 120. This configuration also helps prevent torsional loading on blade 170.

Referring now to FIGS. 2 and 5, gear box 90 may further include a slot 92 defined therein corresponding to slot 29 defined within shaft 12a, such that finger tab 43 may extend from gear box 90 and shaft 12a. Additionally, gear box 90 may extend along a substantial length of the racks 50, 60, or just a portion of the racks 50, 60, depending on the load expected to be encountered by the blade 170, i.e., depending on the size and composition of tissue 400 to be cut. Further, as shown in FIG. 5, gear box 90 may extend further along one of the racks, e.g., actuator rack 50, to provide additional support.

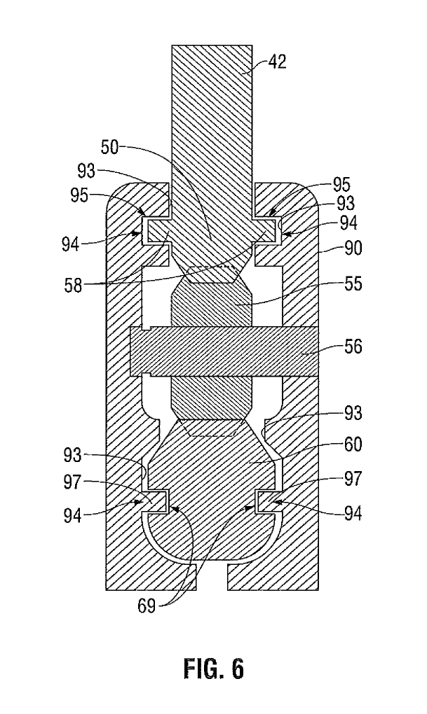

Referring now to FIG. 6, gear box 90 may include a set of guide tracks 94 defined therein for maintaining the fixed spatial relationship between pinion gear 55, actuator rack 50, and blade rack 60. More specifically, gear box 90 may include a set of recesses 95 extending longitudinally along an inner wall 93 of gear box 90. Actuator rack 50 may include a pair of complementary-shaped protrusions, or flanges 58, extending outwardly from opposite longitudinal sides thereof for engagement within the recesses 95 defined within gear box 90. The engagement of recesses 95 of gear box 90 and flanges 58 of actuator rack 50 maintains the spatial relationship between rack 50 and gear 55, while inhibiting movement of rack 50 in all but the longitudinal direction. Similarly, recesses 69 may be defined within blade rack 60 and protrusions 97 may extend inwardly from inner wall 93 of gear box 90 such that blade rack 60 translates along guide tracks 94. Although actuator rack 50 is shown including flanges 58 engaging recesses 93 and blade rack 60 is shown including recesses 69 having protrusions 97 engaged therein, it is envisioned that other, similar configurations may employed, e.g., recesses (not shown) of rack 50 may engage protrusions of guide track 94 and flanges (not shown) of rack 60 may engage with recesses of guide track 94. Guide tracks 94 may be configured in any other suitable fashion to engage the actuator rack 50 and/or blade rack 60 and guide the longitudinal translation thereof through the gear box 90.

In an embodiment where gear box 90 includes guide tracks 94, it becomes less important that gear box 90 be made from a relatively rigid material, e.g., metal. Although a metal gear box 90 would add to the strength of gear box 90, it is envisioned that guide tracks 94 provide support to prevent torsional loading on blade 170 and help ensure proper alignment of blade 170 during translation from the retracted position to the extended position. In other words, gear box 90, having guide tracks 94 therein, may, but need not, be formed form metal.

As shown in FIGS. 2 and 4, a biasing spring 33 (or other biasing member) may be employed within chamber 28 of shaft 12a to bias the actuator rack 50 upon proximal movement thereof such that upon release of finger tab 43, the force of spring 33 automatically returns actuator rack 50 to a distal end 29a (FIG. 1) of slot 29 of shaft 12a. A return spring 35, in place of, or in conjunction with, biasing spring 33, may be operatively connected to bias blade rack 60 to achieve the same purpose.

Finger tab 43 includes one or more ergonomically friendly features which enhance the tactile feel and grip for the user to facilitate actuation of the finger tab 43. Such features may include, raised protuberances, rubber inserts, scallops and gripping surfaces and the like.

Forceps 10 may also include a lockout mechanism (not shown) for preventing accidental reciprocation of blade 170 through blade channel 140. Such a feature would prevent blade 170 from being translated distally until the jaw members 110 and 120 are disposed in the closed position. The lockout mechanism may include mechanical components and/or electrical components, such as a sensor.

With reference now to FIGS. 1-6, the operation of forceps 10 is described. Initially, forceps 10 is positioned such that jaw members 110 and 120 are spaced-apart relative to one another with tissue 400 disposed therebetween. At this point, the lockout mechanism (not shown) may prevent inadvertent deployment of blade 170 until jaw members 110 and 120 are moved to the closed position. Once positioned as desired, a user may engage finger holes 18a and 18b to squeeze shafts 12a and 12b together, such that jaw members 110 and 120 are moved from the spaced-apart to the closed position, grasping tissue 400 therebetween. As discussed above, ratchet 30 may selectively lock the jaw members 110 and 120 relative to one another at various positions during pivoting, such that the desired force may be applied accurately and consistently to tissue 400. The user may then selectively apply electrosurgical energy to electrically conductive sealing plates 112 and 122 of jaw members 110 and 120, respectively, to thereby effectuate a tissue seal. Once tissue has been adequately sealed, the user may translate finger tab 43 of actuator 42 proximally, thereby moving actuator rack 50 proximally, rotating pinion gear 55 clockwise and advancing blade rack 60, and thus blade 170, distally through blade channel 140 defined within jaw members 110 and 120 to cut tissue 400 therebetween. When blade 170 has been advanced sufficiently through blade channel 140 to cut tissue disposed between jaw members 110 and 120, finger tab 143 may be released by the user. Under the bias of spring 33 and/or return spring 35, finger tab 43 returns to the initial, distal position, while blade 170 returns to the initial, proximal position disposed within shaft 12a. Once tissue 400 has been sealed and cut, the user may move the finger holes 18a and 18b apart from one another to open the jaw members 110 and 120 such that forceps 10 may be removed from the surgical site.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A forceps comprising:
first and second shaft members each having a jaw member disposed at a distal end thereof, at least one of the jaw members moveable from an open position to a closed position for grasping tissue therebetween, at least one of the jaw members including a blade slot extending longitudinally therethrough, the blade slot configured for reciprocation of a blade therethrough; and
an actuation assembly disposed within one of the shaft members, the actuation assembly configured for selectively translating the blade between a retracted position and an extended position wherein the blade extends at least partially through the blade slot in the extended position, the actuation assembly including:
an actuator mechanically engaging an actuator rack;
a gear member mechanically coupled to the actuator rack, the gear member having a pin disposed therethrough such that the gear member is rotatable with respect to the pin;
a blade rack mechanically coupled to the gear member, the blade rack having the blade disposed at a distal end thereof; and
a gear box configured to house the gear member and at least a portion of the actuator rack and the blade rack therein, the actuator rack and the blade rack being at least partially longitudinally translatable through the gear box, wherein the gear box maintains the gear member, the actuator rack, and the blade rack in a fixed spatial relation relative to one another.

2. The forceps according to claim 1, wherein translating the actuator proximally translates the blade distally to the extended position.

3. The forceps according to claim 1, wherein the gear box is made from a metal.

4. The forceps according to claim 1, wherein the pin is fixedly engaged within the gear box such that the gear member is rotatably secured therein.

5. The forceps according to claim 1, wherein the gear member includes a plurality of teeth disposed along an outer surface thereof, the gear teeth configured for engagement with teeth disposed along both the actuator rack and the blade rack.

6. The forceps according to claim 1, wherein the gear box includes at least one guide track therein, the at least one guide track configured to guide longitudinal translation of at least one of the actuator rack and the blade rack through the gear box.

7. The forceps according to claim 6, wherein the at least one guide track includes at least one pair of recesses defined within opposing sides of the gear box and wherein at least one of the actuator rack and the blade rack include a pair of protrusions extending from opposite longitudinal sides thereof, the protrusions configured to engage the recesses to thereby guide the translation of the at least one of the actuator rack and the blade rack through the gear box.

8. The forceps according to claim 6, wherein the at least one guide track includes at least one pair of protrusions extending inwardly from opposing sides of the gear box and wherein at least one of the actuator rack and the blade rack include a pair of recesses defined therein along opposite longitudinal sides thereof, the protrusions configured to engage the recesses to thereby guide the translation of the at least one of the actuator rack and the blade rack through the gear box.

9. The forceps according to claim 1, further comprising at least one biasing member for biasing the blade in the retracted position.
